(19) United States
(12) Patent Application Publication
(10) Pub. No.: US 2023/0134811 A1
(43) Pub. Date: May 4, 2023

(54) SYSTEMS AND METHODS FOR SCORE BASED CLUSTERS

(71) Applicant: Welldoc, Inc., Columbia, MD (US)

(72) Inventors: Anand IYER, Columbia, MD (US); Malinda PEEPLES, Columbia, MD (US); Mansur SHOMALI, Columbia, MD (US); Abhimanyu KUMBARA, Columbia, MD (US); Michelle DUGAS, Arlington, VA (US); Guodong GAO, Clarksville, MD (US); Junjie LUO, Baltimore, MD (US)

(73) Assignee: Welldoc, Inc., Columbia, MD (US)

(21) Appl. No.: 18/052,094

(22) Filed: Nov. 2, 2022

Related U.S. Application Data
(60) Provisional application No. 63/263,461, filed on Nov. 3, 2021.

(51) Int. Cl. G16H 20/60; G16H 10/60; G16H 50/30; G16H 40/63

(52) U.S. Cl. CPC G16H 20/60; G16H 10/60; G16H 50/30; G16H 40/63

(57) ABSTRACT

Methods and devices include managing a metabolic metric of a user by determining an activity behavior score for the user based on self-monitoring activity inputs and activity performance. A carbohydrate behavior score for the user may be determined based on self-monitoring carbohydrate inputs and carbohydrate performance. A medicine behavior score may be determined based on user consumption of medicine according to a medicine schedule. A user cluster may be identified from a plurality of clusters that are determined based on receiving initial user activity, carbohydrate and medicine behavior scores for a plurality of initial users. The plurality of user clusters may be generated by applying a clustering algorithm to the initial user activity, carbohydrate, medicine behavior scores. A metabolic metric trend may be determined based on the user cluster and used to generate a treatment plan for the user to improve a metabolic metric outcome.

### RELATED APPLICATIONS

This application claims the benefit of priority to U.S. Provisional Application No. 63/263,461, filed on Nov. 3, 2021, which is incorporated herein by reference in its entirety.

### TECHNICAL FIELD

The present disclosure relates generally to obtaining and processing data to generate treatment plans for users based on user clusters, and, in some embodiments, specifically toward generating targeted outreach and support based on cluster based pattern identification.

### INTRODUCTION

Increased healthcare costs have limited user access to appropriate care. At the same time, healthcare companies have increased provider workloads and limited physician-user interactions. Diabetes treatment often relies on sporadic readings (e.g., glucose readings) that do not provide ample data to effectively provide treatment options. Such readings are often used in isolation such that changes are recommended based on just a few readings. Any medical, dietary, and/or lifestyle changes recommended as a result of a given reading are therefore limited given the sparse data received via the sporadic readings.

The present disclosure is directed to addressing one or more of the above-referenced challenges. The introduction provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

This disclosure is directed to at least a computer-implemented method for managing a relevant metabolic metric associated with a chronic condition of a current user, the method including: determining an activity behavior score for the current user, based on receiving self-monitoring activity inputs and automatically monitoring activity performance via a motion sensor associated with an electronic device; determining a carbohydrate behavior score of the current user, based on self-monitoring carbohydrate inputs and carbohydrate performance; determining a medicine behavior score of the current user, based on the current user's consumption of medicine in accordance with a medicine schedule; identifying a user cluster for the user, from a plurality of clusters, the plurality of clusters determined based on: receiving initial user activity behavior scores, carbohydrate behavior scores, and medicine behavior scores for a plurality of initial users; and generating the plurality of user clusters by applying a clustering algorithm to the initial user activity behavior scores, carbohydrate behavior score, and medicine behavior scores for the plurality of initial users; determining a current user metabolic metric trend for the user based on the identified user cluster; and generating a treatment plan for the current user to improve a metabolic metric outcome based on the current user metabolic metric trend.

Determining the medicine behavior score may include multiplying a percentage of medications consumed as scheduled by a factor of approximately 1.25. A maximum score for the medication score may be approximately 100. Applying the clustering algorithm may include performing k-means clustering and identifying subgroups of patterns in usage across the initial user activity behavior scores, carbohydrate behavior scores, and medicine behavior scores. Determining the activity behavior score may include weighting the self-monitoring activity inputs relative to the activity performance. Determining the carbohydrate behavior score may include weighting the self-monitoring carbohydrate inputs relative to the carbohydrate performance. Applying the clustering algorithm may include: determining initial user total behavior scores based on the initial user activity behavior scores, carbohydrate behavior scores, and medicine behavior scores; applying the clustering algorithm to the initial user total behavior scores, activity behavior scores, carbohydrate behavior score, medicine behavior scores, and initial user inputs by the plurality of initial users such that the initial user inputs include at least one of metabolic inputs and symptom inputs. The initial user inputs may include metabolic inputs including at least one of blood glucose entries, blood pressure entries, weight entries, labs entries, and screenings entries for the plurality of initial users. The initial user inputs may include symptom inputs in a form of annotations, the annotations including at least one of mood-related entries, food-related entries, schedule-related entries, activity-related entries, and medication-related entries for the plurality of initial users.

This disclosure is also directed to at least a computer-implemented method for managing a metabolic metric associated with a chronic condition of a current user, the method including: receiving user data for each a plurality of user data categories, for each of a group of users; receiving a metabolic metric path for each of the group of users; determining a respective success score for each of the plurality of users based on the respective metabolic metric path for each of the group of users; training a machine learning model using a training algorithm, based on the user data and the respective success score for each of the plurality of users, the machine learning model being trained to output a new user success score based on new user data; identifying one or more key user data categories based on training the machine learning model, wherein each of the key user data categories meet a category user threshold to determine the new user success score based on the new user data; determining each of an influence component score, a self-monitoring component score, and a time-independence component score for the one or more key user data categories; determining a predictive value score for each of the one or more key user data categories, based on the respective influence component score, the self-monitoring component score, and the independence component score for the one or more key user data categories; and outputting one or more predictive key user data categories based on the predictive value score for each of the one or more key user data categories, wherein the one or more predictive key user data categories meet a predictive value threshold.

The at least one metabolic metric may include a blood glucose level. The user data may be tagged based on the respective success score for each of the users. Identifying the key user data categories may include determining a causal link relationship between a first user data category and the respective success score of a user associated with the first user data category. The causal link relationship may include one of a one-to-one relationship, an inverse relationship, a proportional relationship, a non-linear relationship, or a logarithmic relationship between a change in user data associated with the first user data category for an evaluation period and the new user success score. At least a subset of the user data may be provided by a digital health application. The training algorithm may be one of a supervised training algorithm or an unsupervised training algorithm. A GUI may be generated and may include a first notification based on a first key user data category of the key user categories and a second notification based on a second key user data category of the key user categories, wherein the first notification and the second notification are ordered based on a first category use value the first key user data category and a second category user value of the second key user data category. The machine learning model may be updated based on the one or more predictive key user data categories.

This disclosure is also directed to at least a system for managing a relevant metabolic metric associated with a chronic condition of a current user, the system including: a memory having processor-readable instructions stored therein; and a processor configured to access the memory and execute the processor-readable instructions, which, when executed by the processor configures the processor to perform a method, the method comprising: determining an activity behavior score for the current user, based on self-monitoring activity inputs and automatically monitoring activity performance via a motion sensor associated with electronic device; determining a carbohydrate behavior score of the current user, based on self-monitoring carbohydrate inputs and carbohydrate performance; determine a medicine behavior score of the current user, based on the current user's consumption of medicine in accordance with a medicine schedule; identifying a user cluster from a plurality of clusters, the plurality of clusters determined based on: receiving initial user activity behavior scores, carbohydrate behavior score, and medicine behavior scores for a plurality of initial users; and generating the plurality of user clusters by applying a clustering algorithm to the initial user activity behavior scores, carbohydrate behavior score, and medicine behavior scores for the plurality of initial users; determining a current user metabolic metric trend based on the user cluster; and generating a treatment plan for the current user to improve a metabolic metric outcome based on the current user metabolic metric trend.

Applying the clustering algorithm may include performing k-means clustering and identifying subgroups of patterns in usage across the initial user activity behavior scores, carbohydrate behavior scores, and medicine behavior scores.

### BRIEF DESCRIPTION OF THE DRAWINGS

The accompanying drawings, which are incorporated in and constitute a part of this specification, illustrate examples of the disclosure and together with the description, serve to explain the principles of the disclosure.

FIG. 1 is a schematic illustration of a health management system, according to an example of the present disclosure.

FIG. 2 is a schematic illustration of a portion of the health management system of FIG. 1.

FIG. 3A is a schematic illustration of another portion of the health management system of FIG. 1.

FIG. 3B is a schematic illustration of training an exemplary machine learning model, according to an example of the present disclosure.

FIG. 3C is a flowchart for generating a treatment plan, according to an example of the present disclosure.

FIG. 4 is a histogram of average activity behavior scores over a time period, according to an example of the present disclosure.

FIG. 5 is a histogram of average carbohydrate behavior scores over a time period, according to an example of the present disclosure.

FIG. 6 is a histogram of average medicine behavior scores over a time period, according to an example of the present disclosure.

FIG. 7 is a histogram of total average behavior scores over a time period, according to an example of the present disclosure.

FIG. 8 shows a graph for determining an optimal number of clusters, according to another example of the present disclosure.

FIG. 9 shows another graph for determining an optimal number of clusters, according to another example of the present disclosure.

FIG. 10 shows initial users distributed over various numbers of clusters, according to an example of the present disclosure.

FIG. 11 shows behavior scores by different clusters, according to an example of the present disclosure.

FIG. 12 is a flowchart of an exemplary method for training an exemplary machine learning model based on identifying success-determinative user data categories, according to an example of the disclosure.

FIG. 13 is a simplified functional block diagram of a computer that may be used to implement techniques disclosed herein, according to an example of the present disclosure.

An Appendix is provided herewith and includes a description with examples of the present disclosure including experimental results.

### DETAILED DESCRIPTION

Reference will now be made in detail to examples of the disclosure, which are illustrated in the accompanying drawings. Wherever possible, the same reference numbers will be used throughout the drawings to refer to the same or like parts.

In the discussion that follows, relative terms such as “about,” “substantially,” “approximately,” etc. are used to indicate a possible variation of ±10% in a stated numeric value. It should be noted that the description set forth herein is merely illustrative in nature and is not intended to limit the examples of the subject matter, or the application and uses of such examples. Any implementation described herein as exemplary is not to be construed as preferred or advantageous over other implementations. Rather, as alluded to above, the term “exemplary” is used in the sense of example or “illustrative,” rather than “ideal.” The terms “comprise,” “include,” “have,” “with,” and any variations thereof are used synonymously to denote or describe a non-exclusive inclusion. As such, a process, method, article, or apparatus that uses such terms does not include only those steps, structure or elements but may include other steps, structures or elements not expressly listed or inherent to such process, method, article, or apparatus. Further, the terms “first,” “second,” and the like, herein do not denote any order, quantity, or importance, but rather are used to distinguish one element from another. Moreover, the terms “a” and “an” herein do not denote a limitation of quantity, but rather denote the presence of at least one of the referenced item.

### Healthcare and Computing Environment

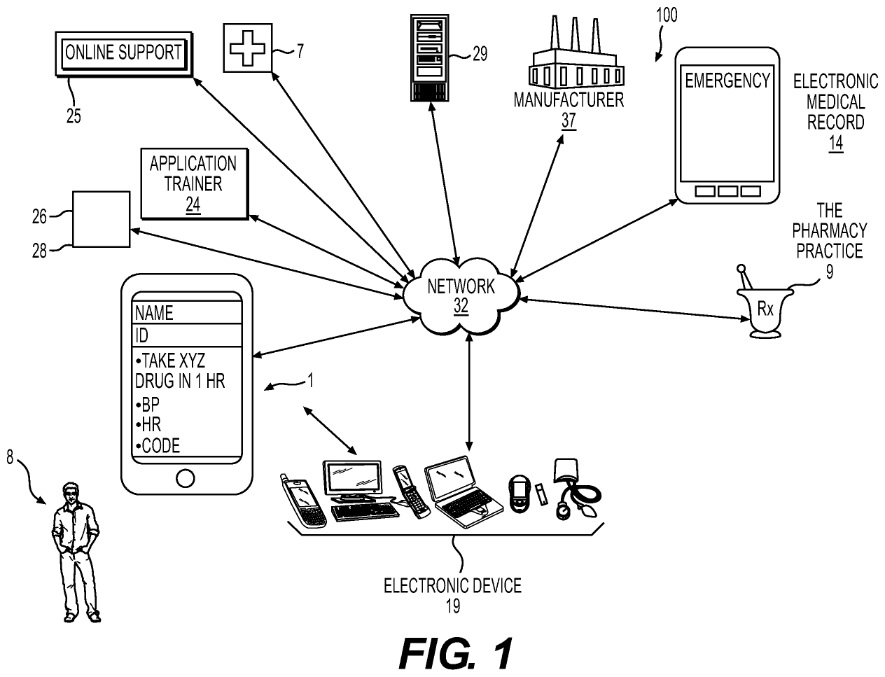

FIG. 1 is a block diagram of a health management system 100, according to an example of the present disclosure. A user (e.g., a patient, consumer, or the like) 8 having an electronic device 19, such as a mobile device, computer, medical device, or any other electronic device configured to access an electronic network 32, such as the Internet, may communicate with or otherwise access a digital health application 1. In some examples, network 32 may include wireless or wired links, such as mobile telephone networks, Wi-Fi, LANs, WANs, Bluetooth, near-field communication (NFC), or other suitable forms of network communication. Multiple electronic devices 19 may be configured to access electronic network 32. A user 8 may access digital health application 1 with a single account linked to multiple electronic devices 19 (e.g., via one or more of a mobile phone, a tablet, and a laptop computer). Electronic device 19 also may include, but is not limited to, mobile health devices, a desktop computer or workstation, a laptop computer, a mobile handset, a personal digital assistant (PDA), a cellular telephone, a network appliance, a camera, a smart phone, a smart watch, an enhanced general packet radio service (EGPRS) mobile phone, a media player, a navigation device, a game console, a set-top box, a biometric sensing device with communication capabilities, a smart TV, or any combination of these or other types of computing devices having at least one processor, a local memory, a display (e.g., a monitor or touchscreen display), one or more user input devices, and a network communication interface. The electronic device 19 may include any type or combination of input/output devices, such as a display monitor, keyboard, touchpad, accelerometer, gyroscope, mouse, touchscreen, camera, a projector, a touch panel, a pointing device, a scrolling device, a button, a switch, a motion sensor, an audio sensor, a pressure sensor, a thermal sensor, and/or microphone. Electronic devices 19 also may communicate with each other by any suitable wired or wireless means (e.g., via Wi-Fi, radio frequency (RF), infrared (IR), Bluetooth, Near Field Communication, or any other suitable means) to send and receive information.

Digital health application 1 may be in communication with other entities or networks to send and receive information. In some examples, digital health application 1 may communicate with one or more applications associated with the user 8 such as, e.g., exercise tracking (e.g., step tracking) applications and/or other health-related applications. Digital health application 1 may be able to import data from the other applications to analyze and use in generating treatment plans for the user 8. For example, digital health application 1 may import activity tracking data from another application and use that data to identify patterns between user 8 exercise and glucose values collected prior to the use of digital health application 1. Digital health application 1 also may import any other suitable data from other mobile health applications such as, e.g., blood pressure, BMI, A1C, exercise type, exercise duration, exercise distance, calories burned, total steps, exercise date, exercise start and stop times, and sleep. Digital health application 1 also may export data to other mobile applications, including, e.g., other mobile health applications having social or interactive features. A healthcare provider 7, such as a physician, may prescribe the application. However, it is also contemplated that digital health application 1 may not require a prescription, e.g., that it may be a commercially available consumer application accessible without a prescription from a digital distribution platform for computer software. Digital health application 1 may be tailored to a specific user 8 and may be activated in person by the user 8 by visiting a pharmacy 9 or other authorized entity. For example, the user 8 may receive an access code from the pharmacy that authorizes access to digital health application 1. The user 8 may receive training on using digital health application 1 by a digital application support system 25 and/or application trainer 24. Digital health application 1 may include programming 28 of various forms, such as machine learning programming algorithms 26. The user treatment plan may include a prescription (e.g., for a drug, device, and/or therapy), which may be dispensed by the pharmacy 9. The pharmacy 9 may allow the refill of the prescribed product/therapy after receiving authorization based on the user's compliance with his/her healthcare treatment plan. The authorization may be received by the pharmacy 9 by a communication from the application 1, via, e.g., the network 32 and various servers 29. Use of the drug or other medical product/therapy also may be sent to the manufacturer 37 over the network 32 to inform the manufacturer 37 of the amount of medical product or therapy being used by user 8. This information may assist the manufacturer 37 in assessing demand and planning supply of the medical product or therapy. The healthcare provider 7 also may receive a report based on the user information received by the application 1, and may update the user treatment plan based on this information. The user's electronic medical record (EMR) 14 also may be automatically updated via the network 32 based on the user information, which may include electronically transmitted user 8 feedback on the application, received by digital health application 1. Healthcare provider 7 may be any suitable healthcare provider including, e.g., a doctor, specialist, nurse, educator, social worker, MA, PA, or the like.

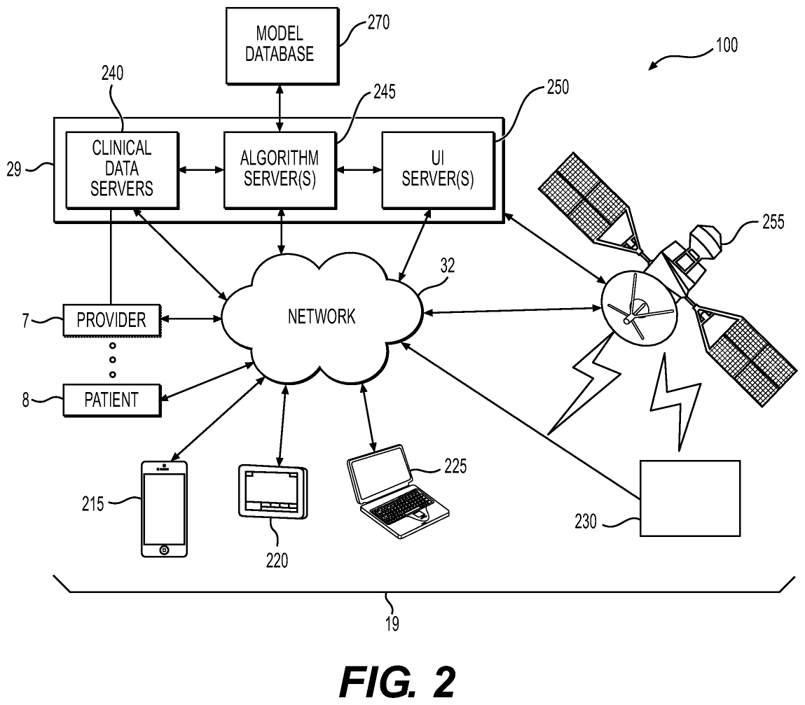

FIG. 2 is a schematic diagram of additional aspects of system 100. For example, the system 100 may access decision models stored on a decision model database 270 via network 32. The retrieved decision models may be used for display and/or processing by one or more electronic devices 19, such as a mobile device 215, a tablet device 220, a computer (e.g., a laptop or desktop) 225, a kiosk 230 (e.g., at a kiosk, pharmacy, clinic, or hospital having medical and/or prescription information), and/or any device connected to network 32.

In the example shown in FIG. 2, mobile device 215, tablet 220, and computer 225 each may be equipped with or include, for example, a GPS receiver for obtaining and reporting location information, e.g., GPS data, via network 32 to and from any of servers 29 and/or one or more GPS satellites 255.

Each of electronic devices 19, including mobile device 215, tablet device 220, computer 225, and/or kiosk 230, may be configured to send and receive data (e.g., clinical information) to and from a system of servers 29 over network 32. Each of devices 19 may receive information, such as clinical data via the network 32 from servers 29. Servers 29 may include clinical data servers 240, algorithm servers 245, user interface (UI) servers 250, and/or any other suitable servers. Electronic device 19 may include a user interface that is in data communication with UI server 250 via network 32. Each server may access the decision model database 270 to retrieve decision models. Each server may include memory, a processor, and/or a database. For example, the clinical data server 240 may have a processor configured to retrieve clinical data from a provider's database and/or a patient's electronic medical record. The algorithm server 245 may have a database that includes various algorithms, and a processor configured to process the clinical data. The UI server 250 may be configured to receive and process user 8 input, such as clinical decision preferences. The satellite 255 may be configured to send and receive information between servers 29 and devices 19.

The clinical data server 240 may receive clinical data, such as data regarding the user from the electronic device 19 via the network 32 or indirectly via the UI server 250. The clinical data server 240 may save the information in memory, such as a computer readable memory.

The clinical data server 240 also may be in communication with one or more other servers, such as the algorithm server 245 and/or external servers. The servers 29 may include data about provider preferences, and/or user 8 health history. In addition, the clinical data server 240 may include data from other users. The algorithm server 245 may include machine learning, and/or other suitable algorithms. The algorithm server 245 also may be in communication with other external servers and may be updated as desired. For example, the algorithm server 245 may be updated with new algorithms, more powerful programming, and/or more data. The clinical data server 240 and/or the algorithm server 245 may process the information and transmit data to the model database 270 for processing. In one example, algorithm server(s) 245 may obtain a pattern definition in a simple format, predict several time steps in the future by using models, e.g., Markov models, Gaussian, Bayesian, PCA (principal component analysis), multi-variate linear or non-linear regression, and/or classification models such as linear discriminant functions, nonlinear discriminant functions, synthetic discriminant functions random forest algorithms and the like, optimize results based on its predictions, detect transition between patterns, obtain abstract data and extract information to infer higher levels of knowledge, combine higher and lower levels of information to understand about the user 8 and clinical behaviors, infer from multi-temporal (e.g., different time scales) data and associated information, use variable order Markov models, and/or reduce noise over time by employing slope-based and curve smoothing algorithms, clustering algorithms, such as k-means clustering.

Each server in the system of servers 29, including clinical data server 240, algorithm server 245, and UI server 250, may represent any of various types of servers including, but not limited to, a web server, an application server, a proxy server, a network server, or a server farm. Each server in the system of servers 29 may be implemented using, for example, any general-purpose computer capable of serving data to other computing devices including, but not limited to, devices 19 or any other computing device (not shown) via network 32. Such a general-purpose computer can include, but is not limited to, a server device having a processor and memory for executing and storing instructions. The memory may include any type of random access memory (RAM) or read-only memory (ROM) embodied in a physical storage medium, such as magnetic storage including floppy disk, hard disk, or magnetic tape; semiconductor storage such as solid-state disk (SSD) or flash memory; optical disc storage; or magneto-optical disc storage. Software may include one or more applications and an operating system. Hardware can include, but is not limited to, a processor, memory, and graphical UI display. Each server also may have multiple processors and multiple shared or separate memory components that are configured to function together within, for example, a clustered computing environment or server farm.

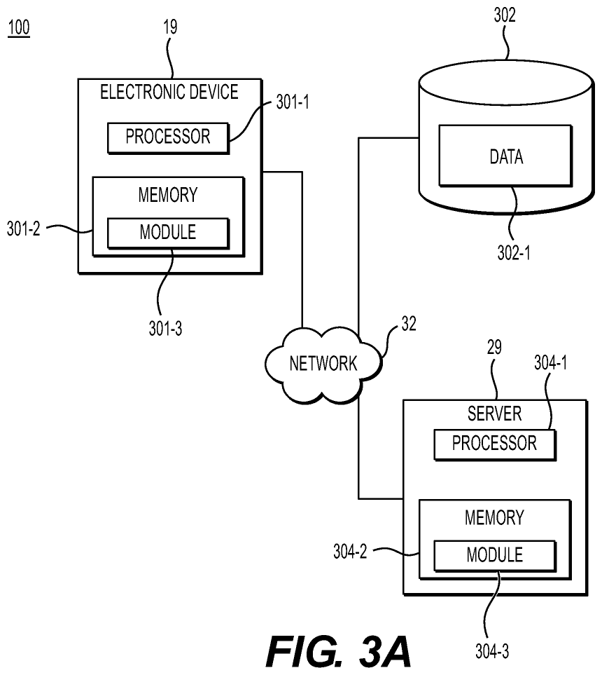

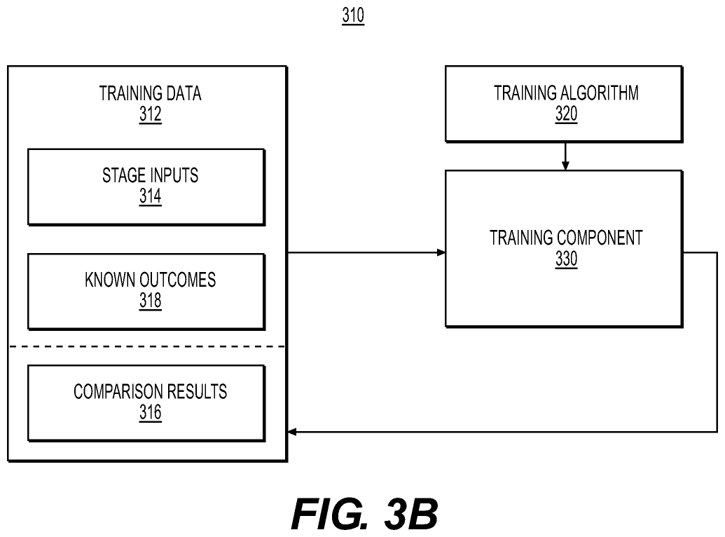

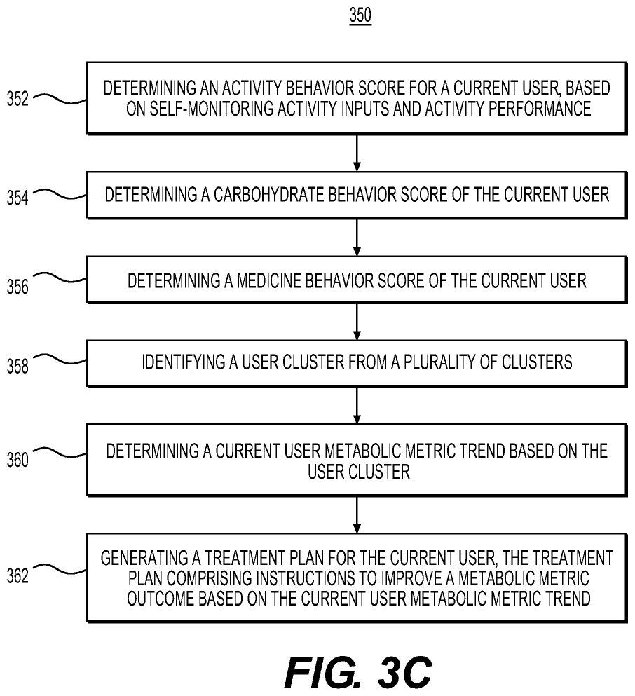

FIG. 3A is another representation of a portion of system 100 showing additional details of electronic device 19 and a server 29. Electronic device 19 and server 29 each may contain one or more processors, such as processors 301-1 and 304-1. Processors 301-1 and 304-1 each may be a central processing unit, a microprocessor, a general purpose processor, an application specific processor, or any device that executes instructions. Electronic device 19 and server 29 also may include one or more memories, such as memories 301-2 and 304-2 that store one or more software modules. Memories 301-2 and 304-2 may be implemented using any computer-readable storage medium, such as hard drives, CDs, DVDs, flash memory, RAM, ROM, etc. Memory 301-2 may store a module 301-3, which may be executed by processor 301-1. Similarly, memory 304-2 may store a module 304-3, which may be executed by processor 304-1.

Electronic device 19 may further comprise one or more UIs. The UI may allow one or more interfaces to present information to a user 8, such as a plan or intervention. The UI may be web-based, such as a web page, or a stand-alone application. The UI also may be configured to accept information about a user 8, such as data inputs and user feedback. The user 8 may manually enter the information, or it may be entered automatically. In an example, the user 8 (or the user's caretaker) may enter information such as when medication was taken or what food and drink the user 8 consumed. Electronic device 19 also may include testing equipment (not shown) or an interface for receiving information from testing equipment. Testing equipment may include, for example, a blood glucose meter, glucose meter, heart rate monitor, weight scale, blood pressure cuff, or the like. The electronic device 19 also may include one or more sensors (not shown), such as a camera, microphone, or accelerometer, for collecting feedback from a user 8. In one example, the device may include a glucose meter for reading and automatically reporting the user's glucose levels.

Electronic device 19 also may include a presentation layer. The presentation layer may be a web browser, application, messaging interface (e.g., e-mail, instant message, SMS, etc.), etc. The electronic device 19 may present notifications, alerts, reading materials, references, guides, reminders, or suggestions to a user 8 via presentation layer. For example, the presentation layer may present articles that are determined to be relevant to the user 8, reminders to purchase medications, tutorials on topics (e.g., a tutorial on carbohydrates), testimonials from others with similar symptoms, and/or one or more goals (e.g., a carbohydrate counting goal). The presentation layer also may present information such as a tutorial (e.g., a user guide or instructional video) and/or enable communications between the healthcare provider, and the user 8, e.g., patient. The communications between the healthcare provider, and the user 8, e.g., patient, may be via electronic messaging (e.g., e-mail or SMS), voice, or real-time video. One or more of these items may be presented based on a treatment plan or an updated treatment plan, as described later. The presentation layer also may be used to receive feedback from a user.

The system 100 also may include one or more databases, such as a database 302. Database 302 may be implemented using any database technology known to one of ordinary skill in the art, such as relational database technology or object-oriented database technology. Database 302 may store data 302-1. Data 302-1 may include a knowledge base for making inferences, statistical models, and/or user information. Data 302-1, or portions thereof, may be alternatively or simultaneously stored in server 29 or electronic device 19.

System 100 can be used for a wide range of applications, including, for example, addressing a user's healthcare, maintaining a user's finances, and monitoring and tracking a user's nutrition and/or sleep. In some implementations of system 100, any received data may be stored in the databases in an encrypted form to increase security of the data against unauthorized access and complying with HIPAA privacy, and/or other legal, healthcare, financial, or other regulations.

For any server or server 29 systems depicted in system 100, the server or server system may include one or more databases. In an example, databases may be any type of data store or recording medium that may be used to store any type of data. For example, database 302 may store data received by or processed by server 29 including information related to a user's treatment plan, including timings and dosages associated with each prescribed medication of a treatment plan. Database 302 also may store information related to the user 8 including their literacy level related to each of a plurality of prescribed medications.

As further disclosed herein, one or more components of the disclosed subject matter may be implemented using a machine learning model. FIG. 3B shows an example training module 310 to train one or more of the machine learning models disclosed herein. It will be understood that a different training module may be used to train each of the machine learning models disclosed herein and/or a single training module 310 may be used to train two or more machine learning models.

As shown in FIG. 3B, training data 312 may include one or more of stage inputs 314 and known outcomes 318 related to a machine learning model to be trained. The stage inputs 314 may be from any applicable source including a healthcare provider 7, one or more servers 29, electronic devices 19, EMR 14, an output from a step. The known outcomes 318 may be included for machine learning models generated based on supervised or semi-supervised training. An unsupervised machine learning model may not be trained using known outcomes 318. Known outputs 318 may include known or desired outputs for future inputs similar to or in the same category as stage inputs 314 that do not have corresponding known outputs.

The training data 312 and a training algorithm 320 may be provided to a training component 330 that may apply the training data 312 to the training algorithm 320 to generate a machine learning model. According to an implementation, the training component 330 may be provided with comparison results 316 that compare a previous output of the corresponding machine learning model to apply the previous result to re-train the machine learning model. The comparison results 316 may be used by the training component 330 to update the corresponding machine learning model. The training algorithm 320 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

### Health Conditions

Diabetes mellitus (commonly referred to as diabetes) may be a chronic, lifelong metabolic disease (or condition) in which a patient's body is unable to produce any or enough insulin, or is unable to use the insulin it does produce (insulin resistance), leading to elevated levels of glucose in the patient's blood. The three most identifiable types of diagnosed diabetes include: pre-diabetes, type 1 diabetes, and type 2 diabetes. Pre-diabetes is a condition in which blood sugar is high, but not high enough to be type 2 diabetes. Type 2 diabetes is a chronic condition that affects the way the body processes blood sugar. Lastly, type 1 diabetes is a chronic condition in which the pancreas produces little or no insulin.

Diabetes generally is diagnosed in several ways. Diagnosing diabetes may require repeated testing on multiple days to confirm the positive diagnosis of a type of diabetes. Some health parameters that doctors or other suitable healthcare providers use when confirming a diabetes diagnosis include glycated hemoglobin (A1C) levels in the blood, fasting plasma glucose (FPG) levels, oral glucose tolerance tests, and/or random plasma glucose tests. Commonly, a healthcare provider is interested in a patient's A1C level to assist in the diagnosis of diabetes. Glycated hemoglobin is a form of hemoglobin that is measured primarily to identify the three-month average plasma glucose concentration that may be used by doctors and/or other suitable healthcare providers include weight, age, nutritional intake, exercise activity, cholesterol levels, triglyceride levels, obesity, tobacco use, and family history.

Once a diagnosis of a type of diabetes is confirmed by a doctor or other suitable healthcare provider, the patient may undergo treatment to manage their diabetes. Patients having their diabetes tracked or monitored by a doctor or other healthcare provider may be treated by a combination of controlling their blood sugar through diet, exercise, oral medications, and/or insulin treatment. Regular screening for complications is also required for some patients. Depending on how long a patient has been diagnosed with diabetes, digital health application 1 may suggest a specific treatment plan to manage their condition(s). Oral medications typically include pills taken by mouth to decrease the production of glucose by the liver and make muscle more sensitive to insulin. In other instances, where the diabetes is more severe, additional medication may be required for treating the patient's diabetes, including injections. An injection of basal insulin, also known as background insulin, may be used by healthcare providers to keep glucose levels at consistent levels during periods of fasting. When fasting, the patient's body steadily releases glucose into the blood to supply the cells with energy. An injection of basal insulin is therefore needed to keep glucose levels under control, and to allow the cells to take in glucose for energy. Basal insulin is usually taken once or twice a day depending on the type of insulin. Basal insulin acts over a relatively long period of time and therefore is considered long acting insulin or intermediate insulin. In contrast, a bolus insulin may be used to act quickly. For example, a bolus of insulin that may be specifically taken at meal times to keep glucose levels under control following a meal. In some instances, when a doctor or healthcare provider generates a treatment plan to manage a patient's diabetes, the doctor creates a basal-bolus dose regimen involving, e.g., taking a number of injections throughout the day. A basal-bolus regimen, which may include an injection at each meal, attempts to roughly emulate how a non-diabetic person's body delivers insulin. A basal-bolus regimen may be applicable to people with type 1 and type 2 diabetes. In addition to the basal-bolus regimen requiring injections of insulin, the treatment plan may be augmented with the use of prescribed oral medications. A patient's adherence to a treatment plan may be important in managing the disease state of the patient. In instances where the patient has been diagnosed with diabetes for more than six months, for example, a very specific treatment regimen must be followed by the patient to achieve healthy, or favorable, levels of glucose. Ultimately, weekly patterns of these medication types of treatments may be important in managing diabetes. A digital health application 1 may recommend treatment plans to help patients manage their diabetes.

### Exemplary Methods

Patient behavior is a critical component of successfully managing diabetes. Accurate feedback about individual self-management behavior may help patients stay on track with diabetes management. Techniques disclosed herein provide standardized scores for self-management of such behavior. Such scores may be used for outreach and decision-making.

According to implementations of the disclosed subject matter, patient-generated health data (PGHD) may be used to summarize a person's self-management behavior using one or more standardized scores. One or more individuals with distinct patterns of self-management behavior may be identified and targeted for outreach and/or support. Techniques disclosed herein may use algorithms and/or machine learning to perform k-means clustering or segmentation for a group of users. A given individual may be placed in an identified cluster using scores generated based on the individual's PGHD. One or more behavior profiles may be associated with each identified cluster and, based on placing an individual in an identified cluster, a behavior profile for the user may be identified. The behavior profile for a given individual may be used to output a treatment plan for placing the individual in an improved metabolic state. Examples provided herein generally refer to managing blood glucose. However, it will be understood that the techniques disclosed herein may be used for managing a relevant metabolic metric associated with any applicable chronic condition (e.g., blood glucose for diabetes, blood pressure for hypertension, weight for obesity, etc.).

An individual's behavior scores may be based on one or more inputs. Such inputs may include one or more of diet inputs, exercise inputs, medication inputs, metabolic inputs, symptom inputs, and/or the like. These inputs may be collectively referred to as user inputs. Any of the inputs disclosed herein may be provided by user 8 via an electronic device 19. A given input may be auto-generated or may be manually input by user 8 using electronic device 19.

Diet inputs may include carbohydrate entries, image based food detection and/or macro-nutrient entries, food database entries, restaurant locator menu-driven entries, meal-planning macro-nutrient entries, ketone entries, and/or the like, or a combination thereof. Carbohydrate entries may be input by user 8 (e.g., a user may select a food and food amount and/or input a number of carbohydrates consumed). Image based food detection may be implemented by capturing a food item (e.g., via a camera) such that the captured food item is identified via a database using, for example, a machine learning model. The identified food item and its quantity may be translated into a carbohydrate amount. Food database entries may be entered using, for example, captured food items and/or user 8 input. User 8 may select food items from a food database provided to user 8. Restaurant locator menu-drive entries may be identified using a Global positioning system (GPS) sensor (e.g., at an electronic device 19) to identify a restaurant. Based on identifying the restaurant, a restaurant menu may be provided such that food items may be selected from the restaurant menu and their corresponding carbohydrates may be recorded.

Activity inputs may include wearable device entries, user 8 entries, exercise equipment data, gym data, and/or the like. Wearable device entries may be received from electronic device 19, and/or may be received automatically or based on user 8 action. A wearable device may track user 8's activity and may automatically record exercise data. The activity data may be collected by the wearable device automatically or based on user 8 action to begin collecting exercise data. Similarly, exercise equipment data may be collected based on user 8's use of one or more exercise equipment. The exercise equipment may be associated with user 8 (e.g., via a user account) or an electronic device 19 may instruct a given exercise equipment of user 8's identity. Accordingly, the exercise equipment may provide user 8's exercise data based on user 8's use of the exercise equipment. Gym data may be recorded at an exercise facility, class, or other location and may be stored at a gym server. The gym data may be based on exercise equipment used by user 8, at the gym, classes associated with user 8 (e.g., classes that user attends, as recorded via a gym system), or the like.

Medication inputs may include prescription entries (e.g., from a patient or via an application such as a pharmacy application or a prescription application), medication device entries, medical provider entries, and/or the like. Medication devices may provide output based on user 8's use of the medication devices. The output may be triggered upon use of the medication device and may be provided via a local connection (e.g., a Bluetooth connection to an electronic device 19) or via a network connection. Medical provider portal entries may be received from a medical provider portal, where the medical provider portal may include information regarding prescribed medicines and/or consumed medicines.

Metabolic inputs may include blood glucose entries, blood pressure entries, weight entries, labs and/or screenings, or the like. Metabolic inputs may be received based on user 8 input and/or via local or network connections. For example, a blood glucose, blood pressure, or weight entry may be received directly from a medical device used to collect the blood glucose or blood pressure. Labs and/or screenings may include A1C figures, cholesterol figures, triglycerides, exam results, and/or the like and may be input by a lab result platform or by user 8.

Symptom inputs such as annotations may include mood-related entries, food-related entries, schedule-related entries, activity-related entries, medication-related entries, and/or the like. Symptom inputs may be extracted from electronic devices 19 (e.g., schedule-related entries, activity-related entries, medication-related entries, or the like). Additionally, or alternatively, symptom inputs may be input by user 8 (e.g., via electronic devices 19). It will be understood that symptom inputs may be used as annotations for refining behavior scores. For example, as further discussed herein, an exercise behavior score may be calculated for a user. The exercise behavior score may be based on one or more exercise inputs. The exercise behavior score as well as one or more other behavior scores may be provided to a machine learning model that also receives user 8's symptom inputs. The machine learning model may provide an output based on the behavior scores as well as the symptom inputs.

A blood glucose monitoring device may detect user 8's blood glucose levels. The blood glucose monitoring device may be a spot monitoring device or a continuous glucose monitoring (CGM) monitor. A spot monitoring device may be a device that is not worn by the user and may be used throughout the day to measure user 8's blood glucose via individual readings.

A CGM monitor may be a continuous analyte sensor system that includes any sensor configuration that provides an output signal indicative of a concentration of an analyte. The CGM monitor may sense the concentration of the analyte to determine, for example, glucose values, based on a bodily fluid (e.g., interstitial fluid). The bodily fluid may be accessed through a user's skin. The output signal, which may be in the form of, for example, sensor data, such as a raw data stream, filtered data, smoothed data, and/or otherwise transformed sensor data, may be sent to a receiver, which may be connected to the CGM monitor via a wired or wireless connection and may be local or remote from the sensor. According to implementations, the CGM monitor may include a transcutaneous glucose sensor, a subcutaneous glucose sensor, a continuous refillable subcutaneous glucose sensor, a continuous intravascular glucose sensor, or the like. The CGM monitor may be a compact medical system with one or more sensors that is inserted onto a user 8's abdomen and that includes a small cannula that penetrates the user 8's skin. An adhesive patch may hold the monitor in place. The sensor may sense glucose readings in interstitial fluid on a continuous or semi-continuous basis.

As further described herein, the user inputs disclosed above may be used to predict which behaviors (e.g., interactions with digital health platform 1) can be used to improve user outcomes, to predict such behaviors based on a small amount of data, to apply non-supervised learning to identify user behaviors, to leverage patient-generated health data to summarize a person's self-management behavior with a standardized score, and/or to leverage patient-generated health data to summarize a person's self-management behavior with a standardized score. Techniques disclosed herein may implement the above using one or more machine learning models that receive user inputs to provide the solutions described above.

### Cluster Generation

According to an implementation, a plurality of clusters that group users by user behavior may be generated. The clusters may be generated using unsupervised learning and using data from a plurality of initial users, as further described below. After the clusters are generated based on the plurality of initial users, data from active users may be used to determine which of the plurality of clusters an active user is matched with. Based on identifying the cluster that an active user is matched with, a treatment plan may be provided for the active user to, for example, improve the predicted health outcome for the patient, as identified by the active user's cluster.

A treatment plan may be identified by accessing a library of a plurality of treatment plans. Each treatment plan of the plurality of treatment plans include one or more treatment attributes that may be provided to a user. The treatment attributes may include medication information, diet plans, exercise plans, or the like or a combination thereof. A treatment plan of the plurality of treatment plans may be identified, for example, by a machine learning model. The treatment plan may be identified based on inputs such as the given cluster a user is matched with and/or one or more of a user history, a user medical history, a user diet history, a user blood glucose history, a user exercise history, a physiological input (e.g., heart rate, blood pressure, blood glucose), changes or rates of changes to any of the above, or the like, or a combination thereof.

A machine learning model may output a treatment plan based on, for example, the inputs described above. The machine learning model may be trained based on, for example, supervised data including historical inputs, historical treatment plans, and historical results from implementation of the historical treatment plans. The machine learning model, and any machine learning model disclosed herein, may be trained by adjusting one or more of weights, layers, biases, nodes, synapses, and/or the like of an initial machine learning model, based on input data, supervised learning, unsupervised learning, known outcomes, and/or the like.

Clusters may be generated based on one or more behavior scores for a plurality of initial users. The behavior scores may be based on one or more user inputs. Such inputs may include one or more of diet inputs, exercise inputs, medication inputs, metabolic inputs, symptom inputs, and/or the like.

Behavior scores for a user may be based on activity scores, carbohydrate scores, medicine scores, and/or the like, or a combination thereof. According to an implementation, one or more behavior scores may incorporate both a monitoring component and a performance component. Accordingly, the behavior scores may be based on whether a given item was monitored (e.g., input by a user) and also the performance level associated with that item. Behavior scores may be standardized in any applicable manner (e.g., on a range of 0-100 such that 100 meets or exceeds guidelines).

According to an implementation, an activity behavior score may be generated based on both self-monitoring (e.g., based on self-reporting) and based on performance. According to an implementation, a user may be able to earn up to 45 points out of 100 points over a given time period based on self-monitoring. For example, 15 points may be earned per entry of a qualifying activity. A qualifying activity may be any activity designated as such by a user, an activity from a predetermined list of activities, an activity that meets a threshold (e.g., a minimum calorie burn, a heart rate, a duration, etc.) or a combination thereof. Upon entry of the activity by a user (e.g., as input by a user, by an electronic device 19, or any other applicable technique), the user may be allocated a given number of points (e.g., 15 points). According to an implementation, an automated entry (e.g., by a wearable device) may be verified or confirmed by a user prior to the associated points being allocated.

Additionally, a user may be able to earn up to 55 points out of the 100 points over a given time period based on the user's activity performance. For example, 0.366 points may be allocated for one minute of exercise or 100 steps, up to the 55 total points over the given time period. According to this example, a maximum of 150 minutes may be logged for the given time period, to earn the maximum number of points (i.e., 55 total activity performance points in this example).

As an example, a maximum of 100 points may be allocated to a user as described above (45 points based on self-monitoring and 55 points based on performance), in a calendar week. In this example, a given user may log two activity sessions in the calendar week and, thus, may be allocated 30 self-monitoring points. The user may log a total of 90 minutes of activity during the two sessions. Accordingly, an additional 32.94 points may be allocated to the user based on the performance. As a result, the user may earn a 62.94 activity behavior points for the calendar week.

It will be understood that according to implementations, certain activities or frequency of activities may be allocated a different number of points. For example, an activity with a higher exertion rate may be allocated a greater number of points and an activity with a lower exertion rate may be allocated a lesser number of points.

According to an implementation, a carbohydrate behavior score may be generated based on both self-monitoring (e.g., based on self-reporting) and based on goal based performance. According to an implementation, a user may be able to earn up to 50 points out of 100 points over a given time period based on self-monitoring. For example, 10 points per day may be earned for each day that a user reports her carbohydrate intake. Upon entry of the carbohydrate by a user (e.g., as input by a user, by an electronic device 19, or any other applicable technique), the user may be allocated a given number of points (e.g., 10 points per day). According to an implementation, an automated entry (e.g., by electronic device 19) may be verified or confirmed by a user prior to the associated points being allocated.

Additionally, for performance scoring, a user may be able to earn up to 50 points out of the 100 points over a given time period based on the user's carb range guidelines. A user's carb range guidelines may be specific to the user, specific to a cohort of users associated with the user, or may be general guidelines (e.g., published by a medical entity, a food entity, or the like). For example, 0.5 points may be allocated for each entry that meets a carbohydrate range based on a guideline or a number of points per day (e.g., 10 per day) for meeting a carbohydrate range for a given day. According to this example, a maximum of 100 entries may be logged for the given time period, to earn the maximum number of points (i.e., 50 total carbohydrate range points in this example).

As an example, a maximum of 100 points may be allocated to a user as described above (50 points based on self-monitoring and 50 points based on meeting carbohydrate guidelines), in a calendar week. In this example, a given user may log carbohydrates four days out of a calendar week and, thus, may be allocated 40 self-monitoring points. The user may log a total of 70 carbohydrate entries that meet the user's carbohydrate guideline and may be allocated an additional 35 points (70*0.5) based on the same. As a result, the user may earn a 75 carbohydrate behavior score for the given calendar week.

According to implementations, the self-monitoring points and/or the performance points for an activity behavior score and/or carbohydrate behavior score may be weighted. For example, either the self-monitoring points or the performance points may be weighted based on one or more factors such as a user's attributes, metabolic inputs, symptom inputs, and/or the like.

According to an implementation, a medication behavior score may be generated based on a user's medication consumption. According to an implementation, a user may be able to earn up to 100 points for consuming medication as scheduled. A given user's medication schedule, in including a type and quantity of medication, over a given period of time may be determined. The medication schedule may be determined based on user input, based on using a pharmaceutical portal or medical portal, based on a medical provider input, or the like. Given a medication schedule, a determination may be made regarding a percentage of medications taken, as scheduled, over the given period of time. The percentage of medications taken, as scheduled (e.g., 40%) may be multiplied by a factor of 1.25 to determine a medication behavior score. The medication behavior score may be capped at 100 such that taking medication, as scheduled, 80% of the time may result in the highest possible score (i.e., 80*1.25, resulting in a score of 100). The percentage of medications taken may be determined based on user input or automatically via a medical device output (e.g., via a local network), an electronic device 19 output, or the like. According to an implementation, an automated output (e.g., by electronic device 19) may be verified or confirmed by a user prior to the associated points being allocated.

As an example, a maximum of 100 points may be allocated to a user as described above (based on the user's consumption of medicine based on the user's medicine schedule), in a calendar week. In this example, a given user may take medication in accordance with a medication schedule (e.g., at given times and dosages) 40% of the time in the calendar week. As a result, the user may earn a 50 medicine behavior score for the given calendar week (40*1.25).

FIG. 3C is a flowchart 350 for generating a treatment plan in accordance with an implementation of the disclosed subject matter. At step 352, an activity behavior score may be determined based on self-monitoring activity inputs and activity performance in accordance with the subject matter disclosed herein. For example, a user may provide the self-monitoring activity inputs via electronic device 19. Activity performance may be automatically monitored using a wearable device such as electronic device 19 and may further be based on a motion sensor associated with electronic device 19. At step 354, a carbohydrate behavior score of the user may be determined based on monitoring carbohydrate inputs and carbohydrate performance, as disclosed herein. At step 356, a medicine behavior score of the user may be determined based on the current user's consumption of medicine in accordance with a medicine schedule. At step 358, a user cluster, for the user, from a plurality of clusters may be identified. The plurality of user clusters may be generated based on receiving initial user activity behavior scores, carbohydrate behavior scores, and medicine behavior scores for a plurality of initial users and generating the plurality of user clusters by applying a clustering algorithm to the initial user activity behavior scores, carbohydrate behavior score, and medicine behavior scores for the plurality of initial users. The user cluster for a user may be identified in accordance with the techniques disclosed herein. At step 360, a current user metabolic metric trend for the user may be determined based on the identified user cluster. At 362, a treatment plan for the current user may be generated, the treatment plan may include instructions to improve a metabolic metric outcome based on the current user metabolic metric trend. The treatment plan may be output via a GUI, where the GUI may provide treatment notifications that are ordered based on the identified user cluster.

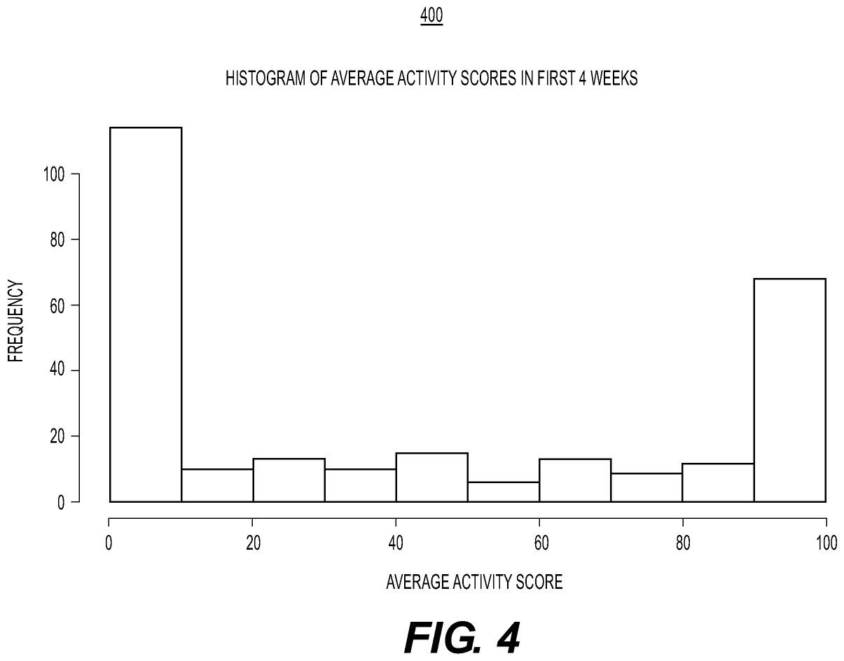

FIG. 4 shows exemplary experimentation results including a distribution 400 of average activity scores over a span of 4 weeks for 270 users. As shown, the distribution 400 of activity scores is a bimodal distribution such that a large group of users captured low activity scores and another large group of users captured high activity scores.

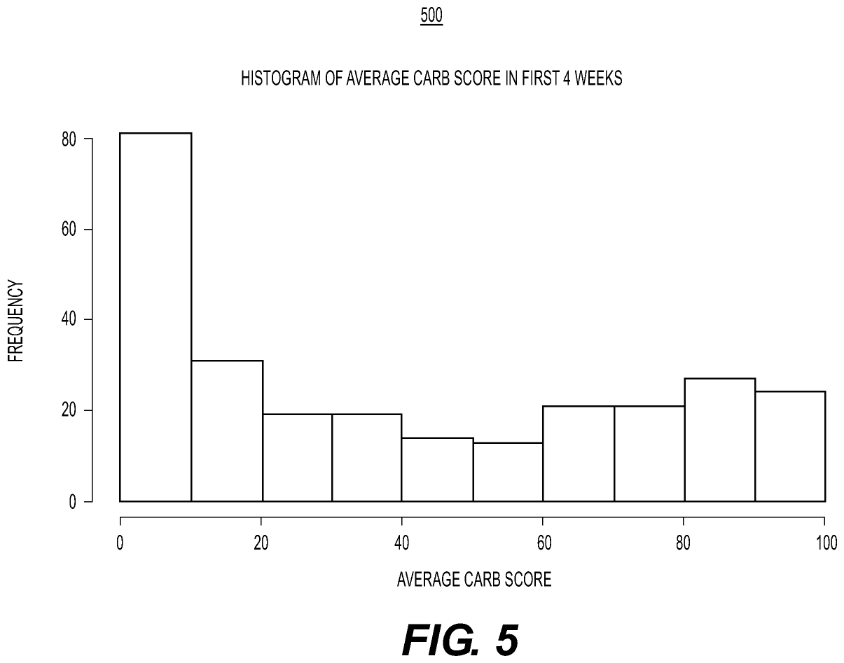

FIG. 5 shows exemplary experimentation results including a distribution 500 of average carbohydrates scores over a span of the same 4 weeks as shown in FIG. 4, for the same 270 users. As shown, the distribution 500 of carbohydrate scores is skewed towards a lower average score per week, such that a large group of users captured low carbohydrate scores compared to the substantially even distribution for mid to high scores.

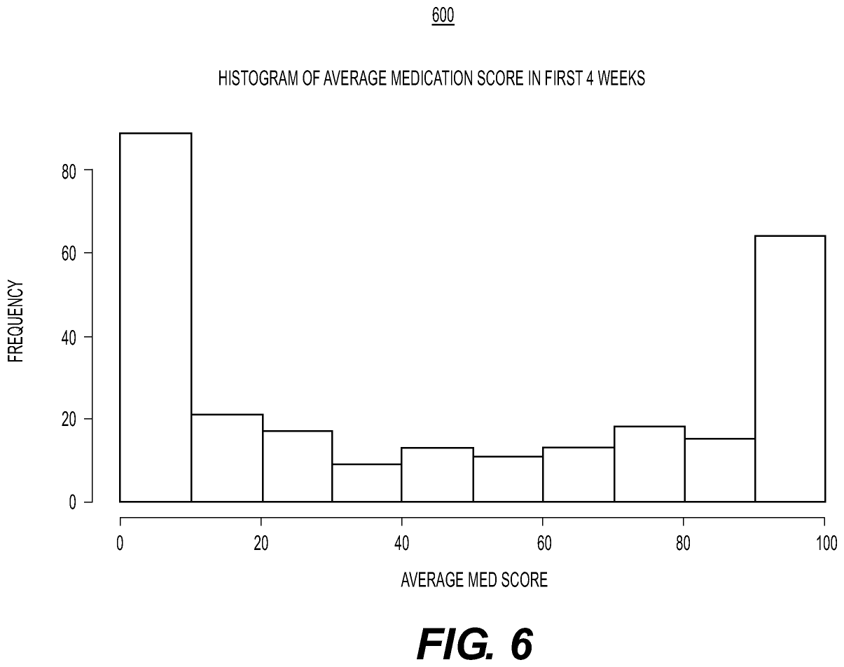

FIG. 6 shows exemplary experimentation results including a distribution 600 of average medicine scores over a span of the same 4 weeks as shown in FIGS. 4 and 5, for the same 270 users. As shown, the distribution 600 of medicine scores is a bimodal distribution such that a large group of users captured low activity scores and another large group of users captured high activity scores. In comparison to the bimodal distribution for the distribution 400 of average activity scores, the bimodal distribution for the distribution 600 of medicine scores is more moderate (less distributed at the two high and low modes).

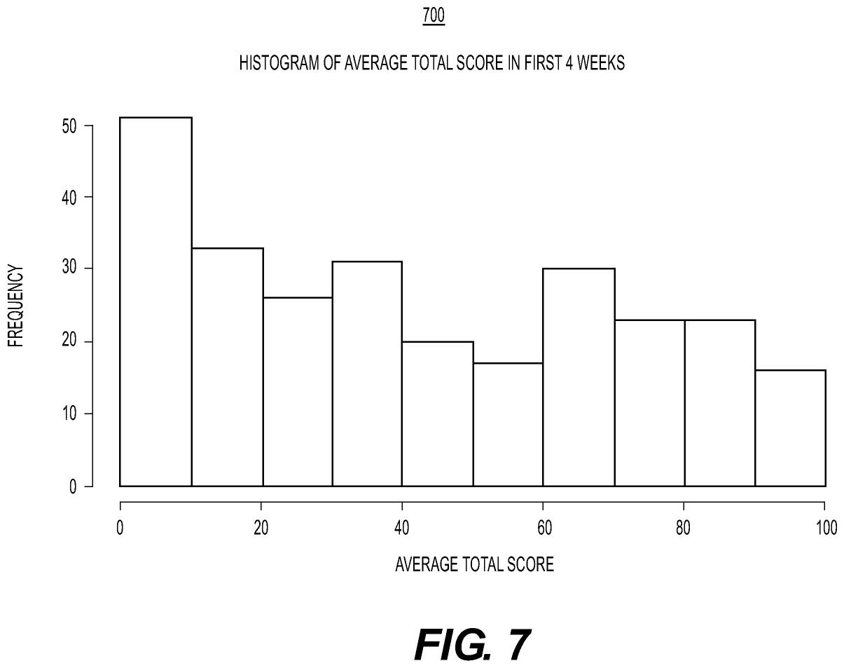

FIG. 7 shows exemplary experimentation results including total scores 700 that may be generated based on the activity scores of FIG. 4, the carbohydrate scores of FIG. 5, and the medicine scores of FIG. 6. According to the example shown in FIG. 7, a total score for a user may be generated by adding the user's activity, carbohydrate, and medicine scores and dividing by the number of scores (e.g., three different scores in this example). The total scores 700 in FIG. 7 may be representative of users' overall scores and may be used in combination with the individual scores (e.g., activity, carbohydrate, and medicine scores) as well as user inputs such as metabolic inputs and/or symptom inputs, to generate clusters, as further disclosed herein.

Based on the behavior sources and user inputs of initial users, k-means clustering may be performed to identify subgroups of patterns in usage across the behavior scores. The clustering may be performed using any applicable technique such as, but not limited to, centroid-based, distribution-based, density-based, hierarchical, auto-encoders, self-organizing maps (SOM), and/or the like. According to implementations, the clustering may be supervised, unsupervised, or semi supervised.

K means and K nearest neighbor (KNN), as disclosed herein, are forms of unsupervised learning. The clustering may be determined based on an initial set of behavior scores (e.g., from the first eight weeks that the scores are collected), based on user inputs, and/or based on user blood glucose levels during a time period (e.g., average blood glucose during the first four weeks).

The number of clusters to be generated based on the behavior scores, blood glucose values, and/or user inputs may be determined based on, for example, a total within sum of square implementation and/or an average silhouette width implementation.

A within-cluster sum of squares is a measure of the variability of the observations within each cluster. In general, a cluster that has a small sum of squares may be more compact than a cluster that has a large sum of squares. Clusters that have higher values exhibit greater variability of the observations within the cluster.

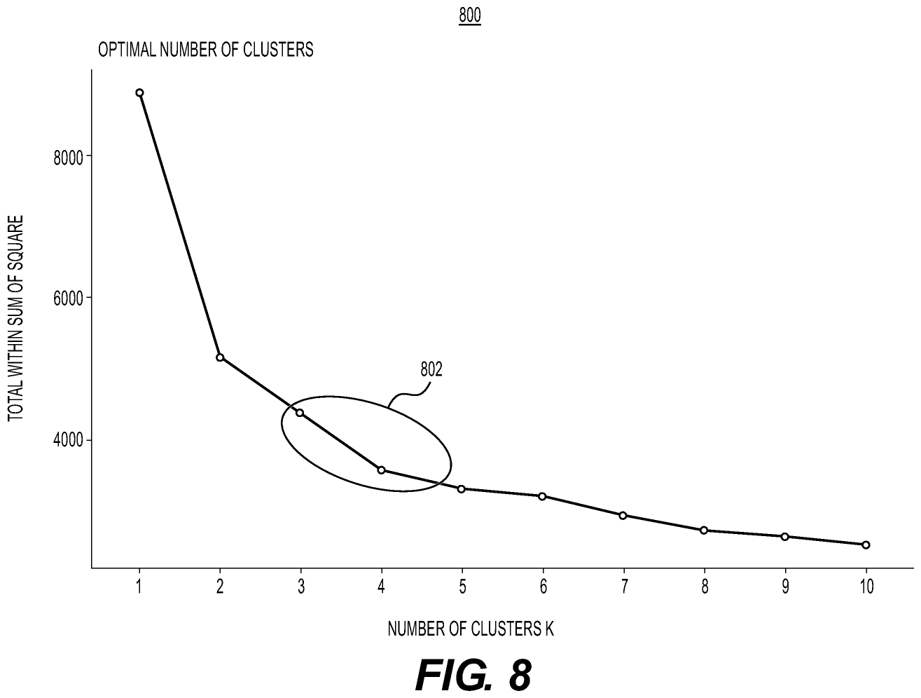

A within-cluster sum of squares implementation may be influenced by the number of observations. As the number of observations increases, the sum of squares becomes larger. Therefore, the within-cluster sum of squares may not directly be comparable across clusters with different numbers of observations. FIG. 8 shows an example chart 800 generated using the within-cluster sum of squares implementation and the behavior scores of FIGS. 4-7.

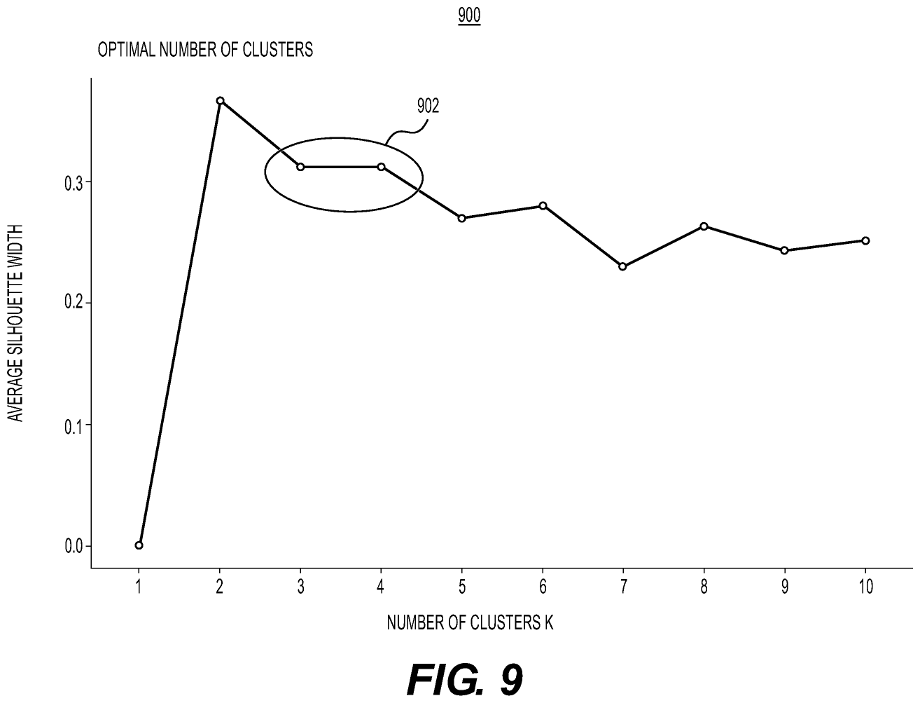

A silhouette implementation refers to a method of interpretation and validation of consistency within clusters of data. The technique provides a succinct graphical representation of how well each object has been classified. A silhouette value may be a measure of how similar an object is to its own cluster (cohesion) compared to other clusters (separation). A silhouette score may range from −1 to +1, where a high value indicates that the object is well matched to its own cluster and poorly matched to neighboring clusters. If most objects have a high value, then the clustering configuration is appropriate. If many points have a low or negative value, then the clustering configuration may have too many or too few clusters. A silhouette may be calculated using any applicable distance metric, such as the Euclidean distance or the Manhattan distance. FIG. 9 shows an example chart 900 generated using the average silhouette width implementation and the behavior scores of FIGS. 4-7.

As shown in FIGS. 8 and 9, the inflection point 802 and the silhouette 902 both indicate that 3 to 4 clusters may be an optimal number of clusters, based on the behavior scores for the 270 users that FIGS. 4-7 are based on. Accordingly, the optimal number of k-means clusters to generate may be either 3 clusters or 4 clusters.

Accordingly, based on the behavior scores FIGS. 4-7, a number of clusters may be generated. The number of clusters may separate each of the 270 users whose behavior scores are shown in FIGS. 4-7, such that each of the 270 users is in a single cluster. As further discussed herein, a given cluster may correspond to users in that cluster having a given blood glucose path. For example, first cluster may correspond to users that are likely to follow an optimal blood glucose path (e.g., where the blood glucose levels of a given user is in an optimal range for optimal durations of time). A second cluster may correspond to users that are likely to follow an unsatisfactory blood glucose path (e.g., where the blood glucose levels of a given user is in outside an optimal range for durations of time).

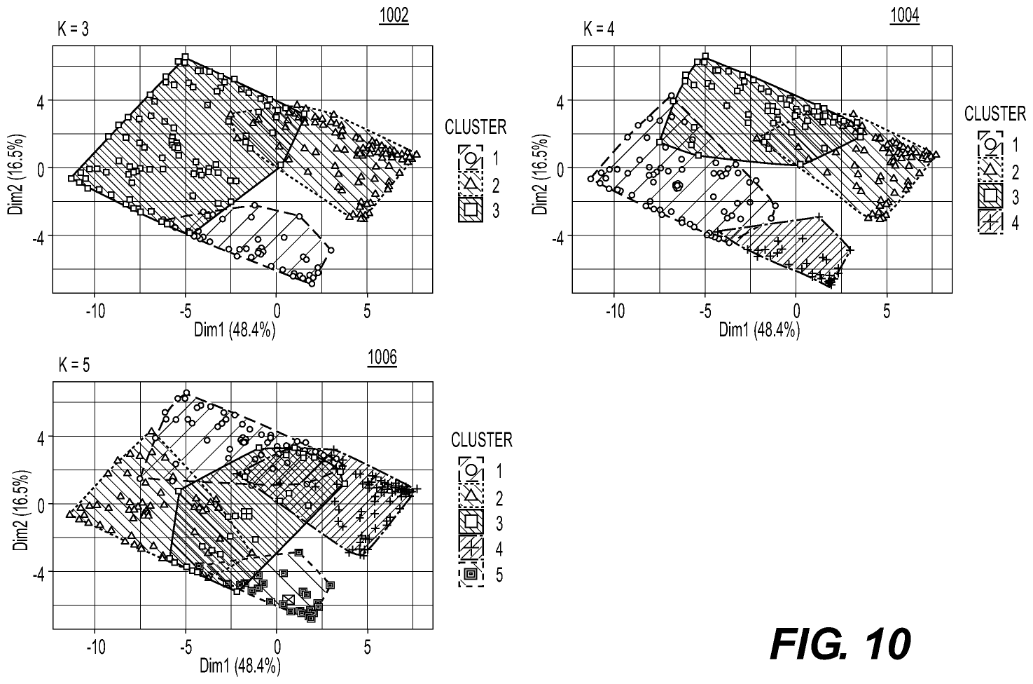

FIG. 10 shows three different clustering arrangements: a first clustering arrangement 1002 with three clusters; a second clustering arrangement 1004 with four clusters; and a third clustering arrangement 1006 with five clusters. In each of the first, second, and third clustering arrangements 1002, 1004, 1006, each of the 270 users may be fit into at least one cluster. As shown, the first clustering arrangement 1002, with three clusters, may have the least number of overlapping users in multiple clusters, whereas the third clustering arrangement 1006, with five clusters, may have the most number of overlapping users. The second clustering arrangement 1004, with four clusters, may have a number of overlapping users between those of the first and third clustering arrangements 1002, 1006. The number of clusters for a given population (e.g., the 270 users whose data is shown in FIGS. 4-7), may be determined based on the within-cluster sum of squares or silhouette techniques shown in FIGS. 8 and 9.

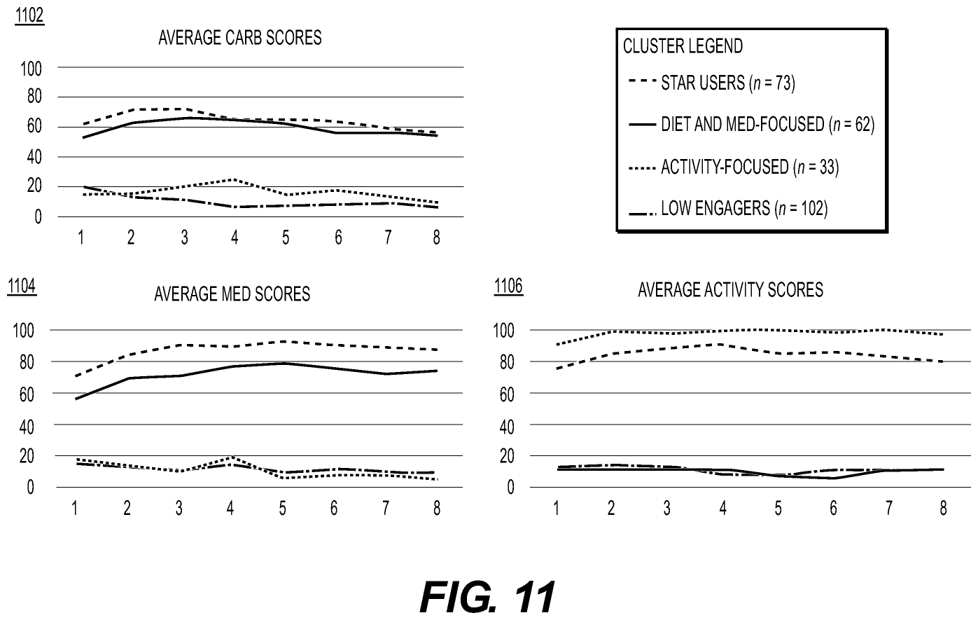

FIG. 11 shows the average behavior scores for the initial users used to generate the clusters shown in FIG. 10. Chart 1102 shows the average carbohydrate behavior scores for users in each of the four respective clusters of the second clustering arrangement 1004. As shown, the highest performing users may be in a start user cluster with an n of 73 and may have an average score indicated by the highest trend line in chart 1102. The star users may be followed by the second highest trend line corresponding to diet and med-focused users with an n of 62. The diet and med-focused trend line may be followed by the next overall highest trend line of activity-focused users with an n of 33. The lowest scoring group may be the low engagers with an n of 102, represented by the overall lowest trend line.

Similarly, chart 1104 includes the average medicine behavior scores for users in each of the four respective cluster arrangements 1004. As shown, the highest performing users may be in the start user cluster with an n of 73 and may have an average score indicated by the highest trend line in chart 1104. The star users may be followed by the second highest trend line corresponding to diet and med-focused users with an n of 62. The diet and med-focused trend line may be followed both the activity-focused users with an n of 33 and the low engagers with an n of 102.

Similarly, chart 1106 includes the average activity behavior scores for users in each of the four respective cluster arrangements 1004. As shown, the highest performing users may be in the activity focused cluster, as their activity scores may overall be the highest. The activity focused cluster may be followed by the start user cluster. The star users may be followed by the diet and med-focused users and the low engagers.

### Cluster Application

Based on the behavior scores for an initial user group (e.g., 270 users according to the experiment disclosed herein), a plurality of clusters (e.g., four clusters) may be generated. The number of clusters may be based on a cluster number optimization algorithm. The cluster number optimization algorithm may determine a number of clusters based on a difference (e.g., an average distance, a mean distance, a mode difference, etc.) between groups of points (e.g., based on an average point of the group of points) and may be further based on a threshold difference. Blood glucose outcomes may be associated with each cluster such that a blood glucose trend prediction for a subsequent user that is placed in a given cluster may be made. The blood glucose outcomes of subsequent user may be used to generate or update a treatment plan for the user. The treatment plan may be provided to the user and may be generated to improve a blood glucose outcome for the user.

The clusters generated based on an initial user group may be applied to a machine learning model during training of the machine learning model (e.g., as disclosed in reference to FIG. 3B).

A current user's behavior scores may be collected over a period of time, as disclosed herein in reference to collecting the behavior scores of the initial users. The current user's behavior scores may be supplemented by user inputs and/or blood glucose values for the current user.

The machine learning model may be trained to receive the current user's behavior scores, user inputs, and/or initial blood glucose values and to place the current user in a cluster. The current user may be placed in a given cluster based on a correlation with the given cluster in comparison to lower correlations with one or more other clusters. The correlations may be determined based on the different behavior scores (e.g., activity behavior score, carbohydrate behavior score, and/or medicine behavior score) and/or the blood glucose values and user inputs associated with the current user. The machine learning model and/or a separate machine learning model may further be configured to generate a blood glucose trend line for the current user and may further be configured to generate a treatment plan based on the trend line.

The treatment plan may be based on the current user's baseline behavior scores, blood glucose values, and/or user inputs. The treatment plan may include goals for behavior scores for a subsequent amount of time. Alternatively or in addition, the treatment plan may include target activities, carbohydrates, and/or medicine consumption on a day to day basis. For example, the treatment plan may be used with digital health application 1, such that digital health application 1 may provide alerts or push notifications to engage in activity, to adjust carbohydrate intake for a given meal or time period, and/or medication consumption triggers.

According to an example, a user may be designated as being associated with the activity-focused track disclosed herein. The activity focused track may not be an optimal blood glucose track. Accordingly, based on determining that the user is associated with the activity focused track, the user may receive daily notifications via her mobile device. The daily notifications may provide a target exercise amount for the day. Notifications may be sent throughout the day at times when the user should consume a given medication. Notifications may also be sent at or around meal times to provide a carbohydrate goal to the user for each given meal. The user may be provided an input option to input a planned meal, and a carbohydrate calculation (e.g., by digital health application 1) may be made. The user may be provided a notification regarding how much of a given type of food is suggested, to meet a carbohydrate requirement for that meal.

### Category-Based Model Training

According to implementations of the disclosed subject matter, user data for a plurality of users may be received. The user data may correspond to a plurality of user data categories such that each user data category has respective user data values. For example, one user data category may be blood glucose levels such that the user data for each of plurality of users may include blood glucose values (e.g., from a CGM monitor that outputs multiple blood glucose values over a period of time). Success scores for each of the users may also be received. The success score for a given user may be based on the user's blood glucose path (e.g., an optimal blood glucose path, an unsatisfactory blood glucose path, etc.). Accordingly, a user's user data may contribute to the user's success core (e.g., blood glucose success score), though it may not be known which user data categories contribute most to the user's success score.

The user data may be provided to a training algorithm to train a machine learning model. The success scores may also be provided to the training algorithm. The training algorithm may be trained to output success scores for new users based on user data for the new users. A new user's success score may correspond to a user cohort and/or a cluster. Alternatively, a machine learning model may output data that indicates a given user cohort and/or a cluster for a new user, based on user data of the new user. The training algorithm and/or a trained machine learning model may also output key user data categories. The key user data categories may be categories that most contributed to determining a success score.

They key user data categories may be further categorized based on influence components, self-monitoring components, and/or independence scores for each given key user data category, as further discussed herein.

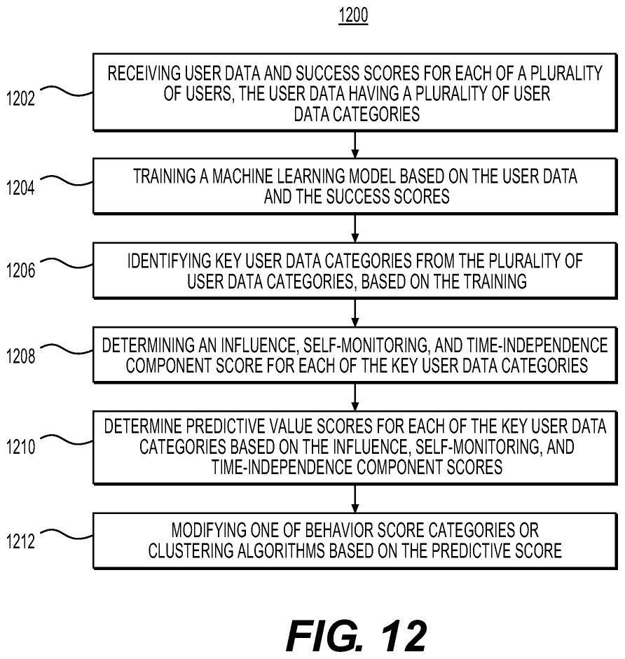

FIG. 12 includes a flowchart 1200 for an implementation of the disclosed subject matter. At step 1202, user data for one or more cohort of users may be received. The user data may be for a cohort of users that exhibit various different blood glucose paths. For example, a first cohort may correspond to users that followed an optimal blood glucose path (e.g., where the blood glucose levels of a given user in the cohort is in an optimal range for optimal durations of time). A second cohort may correspond to users that followed an unsatisfactory blood glucose path (e.g., where the blood glucose levels of a given user is in outside an optimal range for durations of time). Each user may have a corresponding success score, based on the user's blood glucose path, as further discussed herein. The user data for users may be tagged based on each user's respective success score. The users may be categorized based on such tags and may further be categorized as training users or testing users, as further discussed herein.

User data may be received from a user 8, via electronic device 19, from a manufacturer 37, EMR 14, server 29, a health care provider, a network 32 component, and/or the like. The user data may be for one or more user data categories such as demographic data, disease history data, comorbid condition data, detection times (e.g., blood glucose detecting times), survey data, metabolic data, lab data, food data, medication data, activity data, psycho-social and SDOH data, technology familiarity data, engagement data, engagement frequency data, blood glucose data, A1C data, glucose levels, insulin production data, weight data, weight gain or weight loss, heart rate data, resting rate of respiration data, and/or the like. In another example, a user data for one or more users may be derived from labs and/or screenings recorded at the end of an evaluation period, such as the 4 week span from FIGS. 4-7. As previously discussed, such labs and/or screenings may include A1C figures, cholesterol figures, triglycerides, exam results, and/or the like that may have been input at the beginning and the end of the evaluation period by a lab result platform or by a given user that is part of the cohort. In some examples, changes in behavior scores over an evaluation period may be a basis for including a user in a cohort of users whose user data are received at step 1202.

One of ordinary skill in the art will appreciate that user data for a cohort of users may include values for the user data observed at a beginning, an end, and one or more intermediate points within an evaluation period. In yet another example, for a user to be included in the cohort, user may be determined by a comparison between an initial value for a given user data category at the beginning of an evaluation period and an average value for that metric over the evaluation period.

In addition, user data that is tracked may correspond to time periods over which a user may register values for a particular user data category (e.g., blood glucose level) that is within a given range. This type of tracking may be facilitated by a system according to the present disclosure that includes a CGM that monitors a user and takes a high volume of readings each day (e.g., approximately 288 glucose data points). In one example, a user may be included in an above-mentioned cohort where the user's respective user data indicates that a total time per day that a given metric was within a given range increased over the course of the evaluation period. In another example, consecutive periods of time for which user for a user is within a favorable range may be tracked. A user that exhibits an increase in a maximum length of time within a defined increment of time (e.g., hour, day, week) where a given user data category is within a given range, may be included in a cohort of users. For example, over an evaluation period, a user that exhibits an increase of 30 minutes to 35 minutes for a maximum consecutive period of time within a day where the user's blood glucose remains within a given range, may be included in the cohort.

In addition to user data, success scores for each of the users whose user data is received at step 1202 may be received or determined. The success scores may be based on a blood glucose path of each user and may be based on proximity of one or more blood glucose values to an optimal or unsatisfactory blood glucose value, time in range of being in optimal blood glucose values, or the like. As discussed herein, the blood glucose path may be an optimal blood glucose path (e.g., meets a threshold blood glucose value or blood glucose trend) or an unsatisfactory blood glucose path (e.g., does meet a threshold blood glucose value or blood glucose trend).

At step 1204, a training algorithm may be used to train a machine learning model that is trained to output success scores for new users, based on the training using the user data of the users, as received at step 1202. In one example, the training algorithm may be implemented with the user data for each of the users in each of the cohorts of step 1202. In another example, the users may be divided into a training group and a test group. According to this example, the training algorithm may be implemented with the user data from the training group and may be validated using the user data from the test group. In either scheme, implementation of the training algorithm at step 1204 may include training a machine learning model using: (1) the user data; and success score and/or the blood glucose path for each user (or a sub-group thereof). The machine learning model may further be trained using a feedback loop based on user data and corresponding blood glucose paths for those users in the test group.

As discussed herein, user data may correspond to user data categories of tracked or previously recorded data for users in the cohort. The user data categories may include those discussed herein including, but not limited to, blood glucose data, physiological data, demographic data, disease history data, comorbid condition data, detection times (e.g., blood glucose detecting times), survey data, metabolic data, lab data, food data, medication data, activity data, psycho-social determinates of health (PSDOH) data, social determinates of health (SDOH) data, technology familiarity data, actual engagement data, engagement frequency data, or the like. In another example, one or more of the categories of data may include several sub-categories (e.g., demographic data may include age, gender, sex, etc.).

A machine learning model trained in accordance with the techniques above may be trained using a training algorithm such as one or more of a multi-variate regression, K-means classifier, random forest classifier, a neural network model, or the like. In another example, a combination of these training algorithms may be implemented in which output of one model is (A) provided as input for another, or (B) compared to output of another model.

In one example, a training algorithm could employ a supervised training scheme in which user data is compared to success scores (e.g., based on blood glucose paths) using multi-variate regression. In another example, an unsupervised training algorithm may be employed, for example a random forest algorithm, where the machine learning model is trained without using the success scores and/or blood glucose paths but which may be further trained using feedback based on the success scores and/or blood glucose paths. In still another example, both supervised and unsupervised training schemes may be implemented using the user data, success scores and/or blood glucose paths, training user data, testing user data, and or the like associated with the cohorts of users of step 1202.

Accordingly, at step 1204, a training algorithm may be used to adjust one or more of weights, layers, nodes, biases, synapses, or the like, of a machine learning model. The machine learning model may be trained to receive user data of a new user (e.g., new user data) and to output a success score for the user to predict a blood glucose path. The machine learning model may further output or be used to determine a user cohort and/or a user cluster. The success score, user cohort, and/or user cluster may be used to determine a treatment plan for the user, as discussed herein. The machine learning model may further output or be used to determine one or more key user data categories that most contributed to predicting given success scores (e.g., for new users). The one or more key user data categories may be output by training the machine learning model and/or based on the success score output for a new user data. Accordingly, the machine learning model may be used to identify key user data categories that are weighted above a given category use threshold to determine success scores for a given user, a cohort of users, or any new user. The key user data categories may be categories that the machine learning model most relied on to determine the success score.

According to an implementation, the machine learning model may be trained to output success scores based on an initial user data category value (e.g., an initial blood glucose value). For example, the success score output by the machine learning model may be different for a user that starts within an optimal blood glucose range verses a new user who starts within an unsatisfactory blood glucose range.

According to an implementation, the machine learning model may be trained to consider time vectors to determine success scores. For example, the user data for a new user may include actions, physiological states, etc. during multiple input intervals of time (e.g., approximately first 10 days, first 20 days, first 30 days, etc.). The machine learning model may be configured to output success scores at subsequent intervals of times (e.g., approximately 90 days, 180 days, etc.) based on the input interval of times.

In any implementation, training the machine learning model a step 1204 may allow determination of key user data categories most weighted to determine success scores and may include, for example, one or more of demographic data, disease history data, comorbid condition data, detection times (e.g., blood glucose detecting times), survey data, metabolic data, lab data, food data, medication data, activity data, psycho-social and SDOH data, technology familiarity data, engagement data, engagement frequency data, blood glucose data, A1C data, glucose levels, insulin production data, weight data, weight gain or weight loss, heart rate data, resting rate of respiration data, and/or the like. In one example, the machine learning model trained at step 1204 may be used at step 1206 to determine key user data categories that are related to a given success score (e.g., an optimal blood glucose path score, an unsatisfactory blood glucose path score, etc.).

At step 1206, key user data categories based on the machine learning model trained at 1204 may be determined. The machine learning model trained at step 1204 may be used to identify one or more key user data categories from a full set of user data categories included in the user data at step 1202. Accordingly, the key user data categories may be a subset of the user data categories from the full set of user data categories included in the user data. According to an implementation, a first set of key user data categories may be determined to be most indicative of a first success score (e.g., a success score that meets a threshold for an optimal blood glucose path). A second set of key user data categories may be determined to be most indicative of a second success score (e.g., a success score that meets a threshold for an unsatisfactory blood glucose path). Alternatively, according to an implementation, a single set of key user data categories may be indicative of any given success score.

The key user data categories may be determined based on an output of the trained machine learning model that identifies the user data categories having a category use score that meet a category use threshold. The category use threshold may be a value, a use amount, a reliance amount, or the like. The category use threshold may be a threshold that a given user data category may meet, to be considered a key user data category (e.g., a category more relied upon to determine success scores when compared to categories less relied upon to determine success scores). The category use threshold may be a predetermined threshold or a dynamic threshold determined based on the category use values for the user data categories of received user data at step 1202. The category use values and/or category use threshold may be based on weights associated with a user data category, positioning of layers associated with a user data category, or the like.

For example, a machine learning model trained using supervised training, at step 1204, may output which user data categories are weighted the most to predict success or failure (e.g., success scores) relative one or more of the other user data categories of the user data at step 1202. As a result, those key user data categories may be identified at step 1206 (e.g., based on the weight for each of the key user data categories being above a category use threshold, a layer associated with each of the key user data categories being positioned before or after the layers associated with other user data categories, etc.). In another example in which supervised and unsupervised training schemes are implemented, the group of key user data categories may include those user data categories that were weighted higher than a threshold during supervised training and/or also weighted higher than a threshold based on the unsupervised training.

In one example, a key user data category may be identified from the group of user data categories based on a relationship between a change in that key user data category and a change in values for the at least one corresponding user data value and/or any of the success scores. Changes for the user data values may be recognized on a user basis (e.g., changes in food data variable over and evaluation period for a single user) or a user to user basis (e.g., demographic data variable). For example, it may be determined that users in the identified cohort that started an evaluation period with a metabolic metric (e.g., blood glucose) outside of a favorable range, on average, correspond to lower success scores than users started the evaluation period with same metabolic metric (e.g., blood glucose) within a favorable range. As a result, that metabolic metric (e.g., initial blood glucose) may be identified as a key user category.

In another example, it may be determined there is a causal link between a change in the at least one improved key user data category value (e.g., increased exercise), and a change success score. More specifically, it may be observed that a change in the test key user data category value strongly predicts a change in success score. Accordingly, any key user data category from the group of available user data categories determined to have a causal link with respect to success score, may be identified as a key user data category.

At 1208, influence, self-monitoring, and time-independence component scores for select user data categories (e.g., key user data categories or all user data categories) may be determined. The influence, self-monitoring, and time-independence component scores may be a measure of the effect of a given user data category on a success score.

In one example, an influence component score is a measure of a causal link recognized at 1206 for a user data category (e.g., a key user data category). The influence component score may reflect, for example, one-to-one, direct proportionality, inverse proportionality, logarithmic relationship, non-linear relationship, or the like types of relationships between a given user data category and success scores.

The self-monitoring component score may reflect whether or not the select user data category corresponds to an input that can be self-monitored and/or reported. User data categories that cannot be self-monitored (e.g., demographic data) may have a lower self-monitoring component score than those user data categories that can be self-monitored. In the event that a user data category is self-monitored or corresponds to an input that is self-monitored, the self-monitoring component score may reflect whether or not a change in frequency of self-monitoring may have an influence on the success score the user data category is linked to.

The time-independence score is a measure of how changes in data for a user data category occur within an evaluation period affect a causal link with a success score. In one example, the less time-independent a user data category is, the higher the time-independence score may be. Thus, a user data category that has the same impact on a success score, for example, if values for the user data category changes at a beginning, middle, or end of an evaluation period, may have a higher time-independence score than a user data category with an impact that varies relative to when respective data changes occur during an evaluation period.

In another example, a time-independence score may be determined based, in part, on a user data category. A behavior-based user data category may be scored according to whether when a corresponding behavior is performed changes an impact on an improved success score. For example, improvement deltas for overall blood glucose level may be evaluated for: (A) users that test their blood glucose according to a schedule, and for (B) users who test the same number of times as those on a schedule, but do so randomly. The smaller the deviation between the improvement deltas, the higher the time-independence score may be for the corresponding user data category.

In one example, a user data category, even though it is highly time dependent, may be assigned a high time-independence score where step 1208 is performed at a time within an evaluation period for which an impact of the user data category on an improved success score is at a maximum level.

Accordingly, influence, self-monitoring, and time-independence component scores may be used to characterize a given user data category. The influence, self-monitoring, and time-independence component scores for a given user data category may be associated with the given user data category in, for example, database 270.

At step 1210, a predictive value score for each user data category (e.g., each key user data category) may be determined based on the component scores determined at step 1208. The predictive value scores may be standardized or normalized in any applicable manner (e.g., on a range of 0-100 such that 100 meets or exceeds guidelines, a percentage, etc.).

Each of the component scores may have a maximum number of points which that component score can contribute to a predictive value score for a respective user data category. The maximum value each component score may contribute to a predictive value score may be equal (e.g. 33.33 pts out of a 100). In another example, the maximum number of points may vary for the component scores and reflect a weight given to one component score relative to the other component scores.

In still another example, a predictive value score may be determined according to an average of the component scores. In still another example, the component scores may be input into a machine learning model as part of another model production and training implementation. An output from this model may include a weighting scheme for the component scores that may be implemented to determine a predictive score value for a corresponding select variable.

Key user data categories identified at step 1206 may be refined further based on the predictive value scores. For example, key user data categories with a low predictive value score may be removed from a set of key user data categories. Accordingly, predictive key user data categories that meet a predictive value score may be output, and may include a subset of the key user data categories.

At step 1212, behavior scoring and clustering algorithms may be modified based on the predictive value scores for the user data categories determined at step 1210. In one example, the predictive value scores may be utilized to identify updated behavior score categories or and/or clustering algorithms that may replace, be added to, supplement, or modify the behavior score categories (i.e., activity, medicine, and carbohydrate) used to determine the behavior scores as in FIG. 7.

In another example, the predictive value scores, the data for the key user data categories, and/or the data used at 1204 may be combined into training data that is provided with another training algorithm to a training component. The training component may apply the training data to the training algorithm to generate a new machine learning model that utilizes one or more of the key user data categories to score users. Subsequently, these scores can be used to determine comparison results for re-training the new machine learning model.

According to an implementation, key user data categories may be used to generate GUI notifications for a user to reach and/or maintain an optimal blood glucose path. The GUI notifications may be arranged in order based on a key data category's category use value. A user may receive a plurality of notifications via electronic device 19. The notifications may be arranged in order of the key user data category having a highest category use value to the key user data category having the lowest category use value. For example, a first key user data category having the highest category use value may be exercising. Accordingly, a first notification based on a treatment plan that prioritizes exercising at given times, based on the user data, may be prioritized via a GUI. A second key user data category having the next highest category use value may be diet compliance. Accordingly, a second notification based on a treatment plan that prioritizes meal times, based on the user data, may be provided below or subsequent to the first notification, via a GUI.

According to an example implementation, the techniques disclosed herein may be used to train a machine learning model (e.g., via supervised, unsupervised, or a combination of supervised and unsupervised training). As discussed herein, the trained model may output or may be used to determine one or more key user data categories. Further, a new user's user data may be input at the trained machine learning model (e.g., an N+1th user, where N may correspond to a training set of users and/or a previous user whose user data is used to train the machine learning model). Based on the new user data input, the machine learning model may output one or more of a cluster, a cohort, or a success score for the new user. The cluster or cohort may correspond to a group that machine learning model output designates for the new user. The success score may be a score or one or more scores for the new user and may be indicative of the new users metabolic metric path (e.g., blood pressure path).

Based on the cluster, cohort, and/or the success score, a treatment plan for the new user may be identified. The treatment plan may include events such as, for example, one or more activities, actions, tasks, medications, timelines, and/or the like. Such events of the treatment plan may be selected from a library of potential events and may be automatically selected based on a success score, cluster, cohort, user data, user history, etc., or may be output by a machine learning model. According to an implementation, the treatment plan may or may not be specific to a given treatment period (e.g., approximately one week, two weeks, one month, and/or the like). Accordingly, the output of a machine learning model disclosed herein may be used to classify a new user and also may further be used to determine a treatment plan for reaching or following an optimal metabolic metric path.

While steps 1202-1212 of FIG. 12 are depicted in a particular order, the principles of the present disclosure are not limited to the orders depicted therein. System Architecture

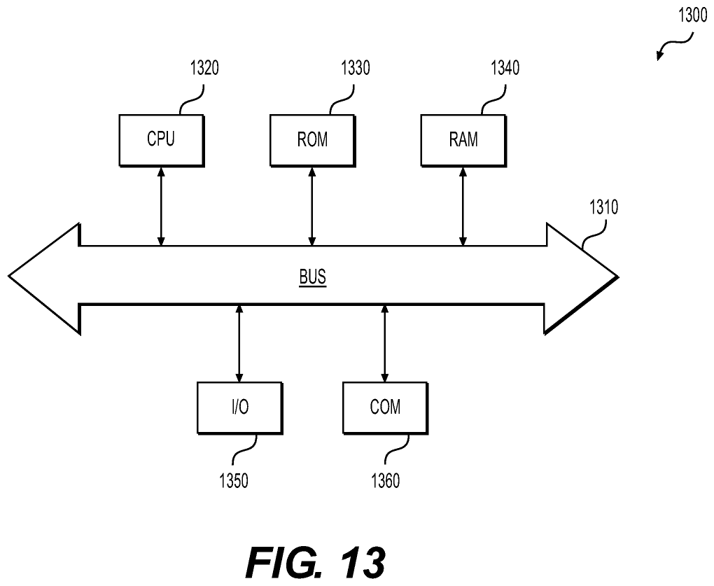

As shown in FIG. 13, a platform 1300 for a server or the like, for example, may include a data communication interface 1360 for packet data communication. The platform also may include a central processing unit (CPU) 1320, in the form of one or more processors, for executing program instructions. The platform typically includes an internal communication bus 1310, program storage, and data storage for various data files to be processed and/or communicated by the platform such as ROM 1330 and RAM 1340 or the like. The hardware elements, operating systems, and programming languages of such equipment are conventional in nature, and it is presumed that those skilled in the art are adequately familiar therewith. The platform 1300 also may include input and output ports 1350 to connect with input and output devices such as keyboards, mice, touchscreens, monitors, displays, etc., and communication interface 1360. Of course, the various server functions may be implemented in a distributed fashion on a number of similar platforms to distribute the processing load. Alternatively, the servers may be implemented by appropriate programming of one computer hardware platform.

It would be apparent to one of skill in the relevant art that the present disclosure, as described herein, can be implemented in many different examples of software, hardware, firmware, and/or the entities illustrated in the figures. Any actual software code with the specialized control of hardware to implement examples is not limiting of the detailed description. Thus, examples are described herein with the understanding that modifications and variations of the examples are possible, given the level of detail presented herein. Aspects of the described subject matter may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

It is to be understood that both the foregoing general description and the following detailed description are exemplary and explanatory only and are not restrictive of the disclosed examples, as claimed.

Other examples of the disclosure will be apparent to those skilled in the art from consideration of the specification and practice of the invention disclosed herein. It is intended that the specification and examples be considered as exemplary only, with a true scope and spirit of the invention being indicated by the following claims.

As is evident from the figures, text, and examples presented above, a variety of embodiments may be contemplated including, but not limited to:

What is claimed is:

1. A computer-implemented method for managing a relevant metabolic metric associated with a chronic condition of a current user, the method comprising:
determining an activity behavior score for the current user, based on receiving self-monitoring activity inputs and automatically monitoring activity performance via a motion sensor associated with an electronic device;
determining a carbohydrate behavior score of the current user, based on self-monitoring carbohydrate inputs and carbohydrate performance;
determining a medicine behavior score of the current user, based on the current user's consumption of medicine in accordance with a medicine schedule;
identifying a user cluster for the user, from a plurality of clusters, the plurality of clusters determined based on:
receiving initial user activity behavior scores, carbohydrate behavior scores, and medicine behavior scores for a plurality of initial users; and
generating the plurality of user clusters by applying a clustering algorithm to the initial user activity behavior scores, carbohydrate behavior score, and medicine behavior scores for the plurality of initial users;
determining a current user metabolic metric trend for the user based on the identified user cluster; and
generating a treatment plan for the current user to improve a metabolic metric outcome based on the current user metabolic metric trend.

2. The method of claim 1, wherein determining the medicine behavior score includes multiplying a percentage of medications consumed as scheduled by a factor of approximately 1.25.

3. The method of claim 2, wherein a maximum score for the medication score is approximately 100.

4. The method of claim 1, wherein applying the clustering algorithm includes performing k-means clustering and identifying subgroups of patterns in usage across the initial user activity behavior scores, carbohydrate behavior scores, and medicine behavior scores.

5. The method of claim 1, wherein determining the activity behavior score includes weighting the self-monitoring activity inputs relative to the activity performance.

6. The method of claim 1, wherein determining the carbohydrate behavior score includes weighting the self-monitoring carbohydrate inputs relative to the carbohydrate performance.

7. The method of claim 1, wherein applying the clustering algorithm includes:
determining initial user total behavior scores based on the initial user activity behavior scores, carbohydrate behavior scores, and medicine behavior scores;
applying the clustering algorithm to the initial user total behavior scores, activity behavior scores, carbohydrate behavior score, medicine behavior scores, and initial user inputs by the plurality of initial users; and
wherein the initial user inputs include at least one of metabolic inputs and symptom inputs.

8. The method of claim 7, wherein the initial user inputs include metabolic inputs including at least one of blood glucose entries, blood pressure entries, weight entries, labs entries, and screenings entries for the plurality of initial users.

9. The method of claim 7, wherein the initial user inputs include symptom inputs in a form of annotations, the annotations including at least one of mood-related entries, food-related entries, schedule-related entries, activity-related entries, and medication-related entries for the plurality of initial users.

10. A computer-implemented method for managing a metabolic metric associated with a chronic condition of a current user, the method comprising:
receiving user data for each a plurality of user data categories, for each of a group of users;
receiving a metabolic metric path for each of the group of users;
determining a respective success score for each of the plurality of users based on the respective metabolic metric path for each of the group of users;
training a machine learning model using a training algorithm, based on the user data and the respective success score for each of the plurality of users, the machine learning model being trained to output a new user success score based on new user data;
identifying one or more key user data categories based on training the machine learning model, wherein each of the key user data categories meet a category user threshold to determine the new user success score based on the new user data;
determining each of an influence component score, a self-monitoring component score, and a time-independence component score for the one or more key user data categories;
determining a predictive value score for each of the one or more key user data categories, based on the respective influence component score, the self-monitoring component score, and the independence component score for the one or more key user data categories; and
outputting one or more predictive key user data categories based on the predictive value score for each of the one or more key user data categories, wherein the one or more predictive key user data categories meet a predictive value threshold.

11. The method of claim 10, wherein the at least one metabolic metric includes a blood glucose level.

12. The method of claim 10, wherein the user data is tagged based on the respective success score for each of the users.

13. The method of claim 10, wherein identifying the key user data categories comprises determining a causal link relationship between a first user data category and the respective success score of a user associated with the first user data category.

14. The method of claim 13, wherein the causal link relationship includes one of a one-to-one relationship, an inverse relationship, a proportional relationship, a non-linear relationship, or a logarithmic relationship between a change in user data associated with the first user data category for an evaluation period and the new user success score.

15. The method of claim 10, wherein at least a subset of the user data is provided by a digital health application.

16. The method of claim 10, wherein training algorithm is one of a supervised training algorithm or an unsupervised training algorithm.

17. The method of claim 10, further comprising generating a GUI comprising a first notification based on a first key user data category of the key user categories and a second notification based on a second key user data category of the key user categories, wherein the first notification and the second notification are ordered based on a first category use value the first key user data category and a second category user value of the second key user data category.

18. The method of claim 10, further comprising updating the machine learning model based on the one or more predictive key user data categories.

19. A system for managing a relevant metabolic metric associated with a chronic condition of a current user, the system comprising:
a memory having processor-readable instructions stored therein; and
a processor configured to access the memory and execute the processor-readable instructions, which, when executed by the processor configures the processor to perform a method, the method comprising:
determining an activity behavior score for the current user, based on self-monitoring activity inputs and automatically monitoring activity performance via a motion sensor associated with electronic device;
determining a carbohydrate behavior score of the current user, based on self-monitoring carbohydrate inputs and carbohydrate performance;
determine a medicine behavior score of the current user, based on the current user's consumption of medicine in accordance with a medicine schedule;
identifying a user cluster from a plurality of clusters, the plurality of clusters determined based on:
receiving initial user activity behavior scores, carbohydrate behavior score, and medicine behavior scores for a plurality of initial users; and
generating the plurality of user clusters by applying a clustering algorithm to the initial user activity behavior scores, carbohydrate behavior score, and medicine behavior scores for the plurality of initial users;
determining a current user metabolic metric trend based on the user cluster; and
generating a treatment plan for the current user to improve a metabolic metric outcome based on the current user metabolic metric trend.

20. The system of claim 19, wherein applying the clustering algorithm includes performing k-means clustering and identifying subgroups of patterns in usage across the initial user activity behavior scores, carbohydrate behavior scores, and medicine behavior scores.
